(19) United States
(12) Patent Application Publication
(10) Pub. No.: US 2006/0116334 A1
(43) Pub. Date: Jun. 1, 2006

(54) Folate based composition for treatment of the cardiovascular system

(76) Inventor: Curt Hendrix, Encino, CA (US)

Correspondence Address: KOPPEL, PATRICK & HEYBL, 555 ST. CHARLES DRIVE, SUITE 107, THOUSAND OAKS, CA, 91360, US

(21) Appl. No.: 11/116,997

(22) Filed: Apr. 27, 2005

Related U.S. Application Data
(63) Continuation-in-part of application No. 11/002,750, filed on Dec. 1, 2004.
(60) Provisional application No. 60/632,681, filed on Dec. 1, 2004.

(51) Int. Cl. A61K 31/714; A61K 31/7048; A61K 31/724; A61K 31/675; A61K 31/525; A61K 31/56

(52) U.S. Cl. 514/27; 514/52; 514/171; 514/251; 514/276; 514/456; 514/350; 514/59; 514/89; 514/58

(57) ABSTRACT

New formulations for the prevention and treatment of cardiovascular diseases arthrosclerosis and risk factors thereof including elevated cholesterol and blood pressure levels comprise phytosterols, flavones and folate, alone or in combination, along with additional compounds chosen to address some or all of the pathways which can result in cardiovascular diseases, namely inflammation, oxidative stress, glycation/dysinsulinemia, platelet function, and homocysteine levels that are important contributors to the development or progression of CVD.

This application is a Continuation-in-Part of Ser. No. 11/002,750 filed Dec. 1, 2004 and claims benefit of Provisional Application 60/632,681 also filed Dec. 1, 2004.

This application is directed to new formulations for improving the health of the cardiovascular system, treating atherosclerosis and other cardiac disease and reducing or preventing risk factors, such as elevated blood pressure, cholesterol levels, particularly LDL-cholesterol, fibrinogen, glycosylation and inflammation which can result in cardiovascular disease (CVD), atherosclerosis and cardiac incidents. These formulations comprise folate in combination with other compounds chosen to address various risk factors and pathways which may lead to these diseases, and particularly in combination with phytosterols and flavones. Also addressed are more readily assimilated forms of phytosterols and flavones.

### BACKGROUND

Folic acid or salts thereof, referred to as folates, along with vitamins B6 and B12 and other biological constituents addressed below, are required for the proper functioning of the metabolic pathways involving methionine, homocysteine, cystathionine, and cysteine. Folate is the generic term for compounds that have vitamin activity similar to pteroylmonoglutamic acid. The term folates as used herein is meant to include all forms of folates including, but not limited to natural and synthetic folic acid, folacin (USP folic acid), naturally occurring folinic acid, 5-methyl tetrahydrofolate, and tetra hydrofolate as well as salts or metabolites of these compounds. FIG. 1 illustrates the various metabolic reactions and constituents of concern. It appears that all three compounds (Folate, B6 and B12) are necessary for normal metabolism. However, these three compounds each function in a different manner. Folate, even if available at normal levels, is consumed in the metabolic process and therefore must be constantly replenished by diet or supplements. However, B6 and B12 function as co-factors. While necessary for the metabolic process to proceed, they are each regenerated in the process. Therefore, if they are present in normal amounts in serum, supplementation may not be necessary. B12 in the form of 5′-deoxyadenosylcobalamin is an essential cofactor in the enzymatic conversion of methylmalonylCoA to succinylCoA. The remethylation of homocysteine (HC) to methionine catalyzed by methionine synthase requires folate (methyltetrahydrofolate) and B12 in the form of methylcobalamin. HC is condensed with serine to form cystathionine (CT) in a reaction catalyzed by cystathionine beta.-synthase which requires B6 (pyridoxal phosphate). CT is also hydrolyzed in another B6-dependent reaction to cysteine and alpha.-ketobutyrate. Homocysteine is a modified form of the amino acid methionine that is tightly regulated by enzymes which require folate. By impairing DNA repair mechanisms and inducing oxidative stress, elevated homocysteine can cause or is a marker of the dysfunction or death of cells in the cardiovascular and nervous systems. Homocysteine appears to be present in many disease states. However, dietary folate stimulates homocysteine removal and may thereby protect cells against disease processes.

The principal biochemical function of folates is the mediation of one-carbon transfer reactions. 5-Methyltetrahydrofolate donates a methyl group to homocysteine, in the conversion of homocysteine to L-methionine. The enzyme that catalyzes the reaction is methionine synthase. Vitamin B12 is a cofactor in the reaction. This reaction, in which folate and vitamin B12 are coparticipants, is of great importance in the regulation of serum homocysteine levels. The L-methionine produced in the reaction can participate in protein synthesis and is also a major source for the synthesis of S-adenosyl-L-methionine (SAMe). The methyl group donated by 5-methyltetrahydrofolate to homocysteine in the formation of L-methionine is used by SAMe in a number of transmethylation reactions involving nucleic acids, phospholipids and proteins, as well as for the synthesis of epinephrine, melatonin, creatine and other molecules. Tetrahydrofolate is the folate product of the methionine synthase reaction. 5-Methyltetrahydrofolate is generated by conversion of 5,10-methylenetetrahydrofolate into 5-methyltetrahydrofolate via the enzyme methyleneterahydrofolate reductase (MTHFR). 5,10-Methylenetetrahydrofolate is regenerated from tetrahydrofolate via the enzyme serine hydroxymethyltransferase, a reaction, which in addition to producing 5,10-methylenetetrahydrofolate, yields glycine.

5,10-Methylenetetrahydrofolate, in addition to its role in the metabolism of homocysteine, supplies the one-carbon group for the methylation of deoxyuridylic acid to form the DNA precursor thymidylic acid. This reaction is catalyzed by thymidylate synthase and the folate product of the reaction is dihydrofolate. Dihydrofolate is converted to tetrahydrofolate via the enzyme dihydrofolate reductase.

Folates are also involved in reactions leading to de novo purine nucleotide synthesis, interconversion of serine and glycine, the metabolism of L-histidine to L-glutamic acid, the metabolism of dimethylglycine to sarcosine and the metabolism of sarcosine to glycine.

One of the natural folates, folinic acid, also known as leucovorin, citrovorum factor and 5-formyltetrahydrofolate, has been used as rescue therapy following high-dose methotrexate in the treatment of osteosarcoma. It is also used to diminish the toxicity of methotrexate, in the treatment of megaloblastic anemia due to folate deficiency and in the prevention or treatment of the toxic side effects of trimetrexate and pyrimethamine. The combination of folinic acid and 5-fluorouracil has until recently been standard therapy for metastatic colorectal cancer. Folinic acid increases the affinity of flurouracil for thymidylate synthase. Folinic acid is available as a calcium salt for parenteral or oral administration.

In addition to being known as pteroylglutamic acid or PGA, folic acid is known chemically as N-[4-[[(2-amino-1,4-di-hydro-4-oxo-6-pteridinyl)methyl]amino]benzoyl]-L-glutamic acid. Older names for folic acid are vitamin B_{g,} folicin, vitamin Bc and vitamin M. Its molecular formula is C₁₉H₁₉N₇O₆ and its molecular weight is 441.40 daltons. Folic acid forms yellowish-orange crystals. The color is imparted by the pteridine ring of folic acid. Pteridine also imparts color to butterfly wings.

Folate has been prescribed as a nutritional supplement for many medical conditions based on the presence of elevated homocysteine levels observed to occur in those conditions. Normal fasting homocysteine levels in adults are generally defined as 5-15 micromoles/L (μmol/L); levels in excess of 100 μmol/L evidence severe homocysteinaemia and are correlated with significantly increased risk of CVD. It has been estimated that exceeding normal levels (5-15 micromol/L) by as little as 5 micromol/L increases the risk of coronary artery disease by 60 percent in men and 80 percent in women. However, it is not clear if these effects are the result of high homocysteine levels or of a folate deficiency which can result in elevated homocysteine, i.e., its presence may denote that it is a marker for a disease condition. Folate supplements appear to reverse the elevated homocysteine levels. However, the elevated homocysteine level may be a result of inadequate supply or excessive consumption of folate and not the cause of the disease. In 1999, and again in November 2000, the FDA found, after an extensive review of the published literature, that lowering homocysteine levels has not been demonstrated to affect vascular disease risk and is not a surrogate marker for vascular disease, and it is not known if elevated levels of homocysteine can cause CVD or whether high homocysteine levels are caused by other factors. However, it is clinically beneficial in such instances to provide folate supplements as individuals who have elevated homocysteine levels appear to be at an increased risk for cardiovascular disease and stroke, and neurodegenerative disorders such as Alzheimer's and Parkinson's diseases as well as neural tube defects, spontaneous abortion, placental abruption, low birth weight, renal failure, rheumatoid arthritis, alcoholism, osteoporosis, neuropsychiatric disorders, non-insulin-dependent diabetes and complications of diabetes, fibromyalgia and chronic fatigue syndrome. According to some researchers, moderate elevations of HC might be associated with increased risk for vascular disease (Ueland et al. (1992) in Atherosclerotic Cardiovascular Disease, Hemostasis, and Endothelial Function (Francis, Jr., ed.), Marcel Dekker, Inc., New York, pp. 183-236). However, folic acid deficiencies (low circulating folate concentrations or low dietary folate intake) are more associated with periphereal vascular disease and increased risk of myocardial infarction, atherosclerotic and coronary disease. This occurs even in individuals with normal homocysteine levels (Bunout, D. et al “Low Serum Folate but Normal Homocysteine Levels in Patients with Atheroslerotic Vascular Disease and Matched Healthy Controls”, Nutrition 2000, 16, p 434-8) suggesting that folates may have a protective effect that extends beyond maintaining normal homocysteine levels and is independent of homocysteine elevation. In addition, increasing folate levels in individuals who had previously experienced a coronary event reduced the likelihood of future coronary events. Moderate hyperhomocysteinaemia has been shown to be frequently present in cases of stroke and to be independent of other stroke risk factors (Brattstrom et al. (1992) Eur. J. Clin. Invest. 22:214-221).

Based on published literature, it is not clear if the various disease states are caused by elevated homocysteine levels or the elevated homocysteine levels are caused by other factors which are the primary cause of the disease state and result in elevated levels of homocysteine. For example, it is also known that folate supplements are usefully where B12 deficiencies exist, but homocysteine levels may not be elevated. Individuals with B12 deficiency can display neurological disorders, typically relating to underlying anemia. However, supplementing diet with only folate is not medically recommended as these folate supplements may mask the underlying B12 problem. U.S. Pat. No. 4,945,083, issued Jul. 31, 1990 to Jansen, entitled Safe Oral Folic Acid-Containing Vitamin Preparation, describes an oral vitamin preparation comprising the combination of 0.1-1.0 mg B12 and 0.1-1.0 mg folate for the treatment or prevention of megaloblastic anemia. Supplementation with vitamin B12, as well as vitamin B2 and lycopene, can provide further reduction of elevated homocysteine levels and B12 reduces the risk of acute coronary events. Vitamin B-5 (pantothenic acid) is also necessary to form acetylcholine.

Normal serum folate levels in healthy individuals are 2.5-20 ng/ml, with levels less than 2.5 ng/ml indicating the possibility of clinically significant deficiency. Like B12 serum levels, however, serum folate levels are a relatively insensitive measure in that only 50-75% of patients with folate deficiency have levels less than 2.5% ng/ml, with most of the remaining 25-50% being in the 2.5-5.0 ng/ml range (Allen (1991), Cecil Textbook of Medicine, 19th Ed.). Daily supplementation with 0.5-5.7 mg/day of folic acid can reduce homocysteine levels by 25% (Brit. Med J., 316, p 894-8 (1998)) and dosages of 15 mg/day can be delivered without apparent toxicity (Boston, A G et al, Kidney Int., 49, p 147-52 (1996)). The recommended daily allowance of folate is 400 g/d. Vitamin B6 dosages of 50-250 mg/day also cause a significant reduction in homocysteine levels caused by methionine ingestion as part of a methionine loading test protocol. B6, in its pyridoxal 5′-phosphate form (PLP or P5P), is essential for taurine synthesis after the formation of homocysteine. Additionally, daily dosages of B6 is associated with decreased C-Reactive Protein.

A series of patents to Allen et al, (U.S. Pat. No. 5,563,126, U.S. Pat. No. 5,795,873, U.S. Pat. No. 6,207,651, U.S. Pat. No. 6,297,224 and U.S. Pat. No. 6,528,496)) teaches the use of oral compositions or a transdermal patch delivering a combination of B12 and folate, or B12, folate and B6, in concentrations sufficient to reduce elevated homocysteine levels by treating either single or multiple deficiencies of B12, folate, and B6. The Allen non-prescription formulations include 0.3-10 mg CN-cobalamin (B12) and 0.1-0.4 mg folate or 0.3-10 mg B12, 0.1-0.4 folate, and 5-75 mg B6. The Allen prescription formulations comprise between 0.3-10 mg CN-cobalamin (B12) and 0.4-10.0 mg folate or 0.3-10 mg B12, 0.4-1.0 mg folate, and 5-75 mg B6.

S-adenosylmethionine (SAMe) is a substance that occurs naturally in the body. A combination of an essential amino acid and ATP, SAMe plays a role in 35-40 biochemical reactions throughout the body. In most people, the body can make all the SAMe it needs, but some individuals have been found to have lower levels of the compound as well as lower levels of folate and vitamin B12. These three substances each play a part in the metabolic process of “methyl donation” or “methylation”, a process in which a molecule comprised of one carbon molecule and three hydrogen atoms is attached to proteins and lipids. After donating the methyl group, SAMe is converted to S-adenosylhomocysteine (SAH) which is then rapidly converted to homocysteine. If the biochemical conditions are correct the homocysteine is then converted back to methionine and SAMe is regenerated. Controlling SAMe production is connected to folate and B12 production and altered levels of B12, folate and SAMe and the resultant existence of homocysteine have been associated with various different disease states, including cardiovascular disease. It has also been found that these methylation reactions are involved in the production of the neurotransmitters serotonin and dopamine in the brain and enzymes that help repair joints and the liver. There is evidence that serotonin is a factor in migraine and is involved in the so called “rebound effect”, because of its vasoconstricting effect when serotonin levels are elevated and subsequent vasodilation as serotonin levels decrease. Whether diseased states are caused by not enough initially available SAMe or the decreased SAMe levels are a consequence of some underlying disease process and the inability of the body to regenerate SAMe is not clear. Coincidently, folate deficiency also appears to reduce brain serotonin. By supplementing the diet with folate, serotonin generation and its metabolism is balanced, and the cycling of vasodilation and vasoconstriction caused by fluctuation in serotonin is minimized.

A factor that contributes to cardiovascular disease, heart attack, stroke and other vascular-related diseases is chronic inflammatory syndrome. Markers of such a condition include elevated fibrinogen, a coagulation factor found in blood, and C-reactive protein (CRP). High fibrinogen levels can induce a heart attack via several mechanisms including platelet aggregation, hypercoagulation and excessive blood thickening. The presence of C-reactive protein increases the risk of destabilized atherosclerotic plaque, which can result in blood flow blockage, and abnormal arterial bleeding. Studies have shown that elevated fibrinogen levels can double the likelihood of a heart attack and high levels of C-reactive protein can triple the likelihood of dying from a heart attack. Individuals with coronary heart disease who had low CRP had a better clinical outcome regardless of LDL-cholesterol levels.

Fibrinogen and C-reactive protein are produced by pro-inflammatory cytokines in the liver (interleukin-1B, interleukin-6 and tumor necrosis factor) as a response to tissue injury, illness, exercise, malignancy or other inflammatory diseases. Individuals with diseased arteries, such as arteries containing aethroslerotic plaque, particularly unstable plaque, have elevated CRP due to the increased presence of inflammatory cells. Cardiovascular disease appears to increase proportionally to the CRP concentration. Elevated levels of CRP appear to be a good predictive marker of a future cardiovascular event. Certain supplements such as DHA fish oil and olive oil, which include omega 3 fatty acids, and DHEA can suppress formation of these cytokines. Omega-3 fatty acids, particularly eicosapentaenoic acid (EPA) and docahexaenoic acid (DHA), have been recognized as having antihypertensive properties. Other extracts, such as nettle leak extract, also show cytokine suppressing properties. Various other agents have also been shown to be useful in inhibiting the platelet aggregation effects of elevated fibrinogen once formed. These include, but are not limited to aspirin, green tea, ginger, gingko, garlic and vitamin E. Alternatively, fibrinogen levels can be reduced by elevating the serum levels of vitamin A, vitamin C in daily dosages in excess of 2000 mg and beta-carotene. On the other hand excessive homocysteine has been indicated to block the natural breakdown of fibrinogen. These elevated homocysteine levels can be reduced by delivery of folic acid, vitamins B6 and B12 and tri-methylglycine (TMG). It also appears that there is a relationship between low vitamin B6 concentrations, particularly the pyridoxal 5′-phosphate (PLP) form of B6, and elevated C-reactive protein and fibrinogen and this is independent of homocysteine levels. Normal or elevated levels of B6 mediate (reduces) the underlying inflammatory process which can lead to cardiovascular disease. Conversely there appears to be a reduced B6 level in individuals having CVD. B6 also functions as an antioxidant, reducing oxidative stress as measured by superoxide radical production, lipid peroxidation and mitochondrial transmembrane potential. PLP appears to be more effective than other forms of vitamin B6 in preventing cholesterol from agglomerating and sticking to blood vessel walls.

Another factor relating to cardiovascular disease is serum cholesterol levels, particularly the total cholesterol and the LDL and triglycerides concentration and the ratio of HDL- to LDL-cholesterol. The cardiovascular risk associated with having high cholesterol levels is well established. Elevated LDL results in the deposit of atherosclerotic plaque and elevated cholesterol levels have been found to interfere with normal endothelial function. The statin drugs, such as atorvastatin (Lipitor), cerivastatin (Baycol), lovastatin (mevacor) pravastatin (Pravachhol) and simvastatin (Zocor) are the primary pharmaceuticals prescribed to treat elevated cholesterol and LDL levels. However, many people with high cholesterol levels either prefer not to take a statin drug, take the drug and get intolerable side effects, or take statins and don't obtain an acceptable cholesterol lowering. Also, statin drugs can lower CoQ10 levels, which can predispose patients to heart disease. The currently accepted goal is to maintain a total cholesterol level below 200 mg/dl and LDL below about 130 mg/dl. If this is to be achieved by people with total cholesterol over 250 mg/dl levels they need an option that is capable of reducing total cholesterol levels by 20-40% (which the statins are capable of doing).

Phytosterols, and phytostanols (which are saturated plant sterols) are cholesterol-like molecules present naturally in small quantities in many fruits, vegetables, nuts, seeds, cereals, legumes, vegetable oils, and other plant sources such as vegetable oils. The presence of phytosterols in diet, normally from about 160 to 450 mg/day, inhibits the absorption of ingested cholesterol in the intestine as well as recirculating endogenous biliary cholesterol, reducing both total cholesterol and LDL levels. Phytosterols have also been shown to reduce the development of atherosclerotic lesions as well as to normalize the coagulation system. Providing phytosterol in an esterified form, such as provided by margarine, in a daily dosage to 2 g/day has been shown to reduce LDL-cholesterol by as much as 10%. Calcium and magnesium have also been shown to decrease serum cholesterol concentrations. When provided along with phytosterols they showed LDL lowering results which were more then merely additive and had a beneficial effect on liver and myocardial hypertrophy and body weight. Published US Application 2003/0133965 is directed to the supplementation of dietary fibers by the addition of phytosterols, folic acid, vitamin B12 in the form of cyanocobalamin, and the pyridoxine form of vitamin B6.

A particular problem with the delivery of an amount of phytosterols effective in reducing cholesterol adsorption from food is that the sterols are not readily water soluble and therefore show a relatively low level of bioavailability through the intestines. Ostland et al found an increased effectiveness of sitostanol in decreasing the absorption of dietary cholesterol if the sitostanol was delivered in a lypholized form as a sitostanol-licithin vessicle (a 50/50 mixture of sitostanol and soy lecithin). (Osterlund, R. E. Jr. et al, Sitostanol Administered in Lecithin Micelles Potently Reduces Cholesterol Absorption in Humans, Am. J. Clin. Nutr., 70, pp 826-831 (1999)).

U.S. Pat. No. 6,312,703 discloses the use of liquid crystal phospholipids (LCP) in the formation of tablets which can incorporate various additives, the lecithin comprises at least 20% of the product weight. Disclosed therein are solid LCP compositions containing tocotrienols, COQ10, bioflavenoids, natural chelating agents, potassium, tocopherol, selenium and statins for cardiovascular applications or containing Omega-3 oils, CoQ10, quercetin, pyncogenol, calcium, magnesium and potassium for the reduction of elevated blood pressure. An example of a nutritional supplement includes 67% LCP and 33% plant sterols.

Flavonoids exert a strong antioxidant activity against the superoxide radical, hydroxyl radical, hydrogen peroxide and lipid peroxide radicals. Sources of flavonoids include hawthorn, ginkgo and bilberry, isolated flavonoids such as quercetin, morin, rutin, gossyretin, chrysin, myricetin, catechins and oligimeric proanthocyanidins, isoflavones such as genistein and daidzein, and particularly citrus polymethoxylated flavones and citrus flavonone glucosides. Citrus polymethoxylated flavones, typically isolated from lemon, lime, tangerine, grapefruit and orange juice or peel, are antioxidants which have also been found to be beneficial in preventing LDL oxidation and reducing serum LDL-cholesterol levels, apolipoprotein B, and diacylglycerol acetyl transferase, suppressing TNFα expression, inhibiting lipid peroxidation, scavenging superoxide anions and hydroxyl radicals and inhibiting platelet aggregation, thus reducing thrombotic tendencies. Clinical and epidemiological studies have shown that flavonoids can reduce cholesterol levels and the risk of heart disease (Hertog, M. G. et al, Lancet, 342, p 1007-1011 (1993)). Soy isoflavones have been shown to reduce cholesterol levels (Kurowska, E. A. et al, J. Nutr., 120, p 831-836 (1990)) U.S. Pat. No. 6,184,246 is directed to a method of inhibiting the generation of cytokines in individuals, particularly the production of TMAα, interlukin-10 and microphage inflammatory protein α, by delivering an effective amount of polymethoxylated flavones. U.S. Pat. Nos. 6,239,114 and 6,251,400 as well as Ser. No. 09/528,488 filed Mar. 17, 2000, now U.S. Pat. No. ______ and published applications 2001/0055627 and 2004/0214882 describe the use of citrus liminoids, flavonoids, certain polymethylflavones and/or tocotrienols (discussed below) for reducing apolipoprotein B, treating atherosclerosis and hypercholesterolemia. Published application 2002/0006953 (abandoned) suggests that monoterpenes, terpenes, and flavonoids increase HDL and reduce LDL serum levels. Published application 2002/0054924 also suggests the use of decharacterized cranberry along with grapefruit flavonoids for lowering cholesterol levels.

U.S. Pat. No. 5,348,974 is directed to the reduction of cholesterol levels, hyperlipidemia and thromboembolic disorders, thus reducing the incidence of cardiovascular disease by the delivery of substantially pure tocotrienols, and particularly synthesized tocotrienols or analogs of tocotrienol. U.S. Pat. No. 4,603,142 is directed the use of d-α tocotrienols for serum cholesterol reduction. Natural tocotrienols, which are related in structure (have an added unsaturated side chain) to vitamin E, can be recovered from cereal grains such as barley, oats, rice, wheat and rye and vegetable oils such as palm oil and rice bran oil. They are antioxidants, anti-inflammatory and halt or slow the deposition of plaque.

Coenzyme Q10 is necessary for the normal functioning of the myocardium and low serum levels of CoQ10 are common in individuals with heart disease. CoQ10 has been used in treating angina, heart failure and the prevention of reperfusion injury after bypass procedures and cardiomyopathy. Delivery of CoQ10 to patients has been shown to reduce the signs and symptoms of congestive heart failure, particularly improve cardiac function, increase cardiac output, and reduce peripheral resistance, both systolic and diastolic blood pressure, heart rate and heart volume and lower serum cholesterol while increasing HDL.

Policosanol, a mixture of essential alcohols isolated from sugar cane, has been found to have a LDL-cholesterol lowering and HDL-cholesterol increasing effect at dosages of up to about 10 mg/day as well as reducing intermittent claudation and platelet aggregation. Higher dosages (20-40 mg/day) appear to also modulate triglyceride levels. When policosanol is delivered in combination with tocotrienols they can reduce plaque formation and atheroma.

Supplementation of the diet with calcium, magnesium, potassium, chromium and selenium can also be beneficially for cardiac health. In individuals who are on diuretics to reduce blood pressure and congestive heart failure, the magnesium and potassium concentration in the body can become depleted. Mg deficiency is believed to be a major risk factor for decreased survival of CHF patients, can trigger cardiac arrhythmias. Mg and Ca supplementation can also be beneficial in reducing blood pressure. The minimum recommended daily requirement for magnesium is 400 mg. Additionally, selenium activates the antioxidant enzyme glutathione peroxidase which has been linked to prevention of coronary events. Chromium, particularly in the form of chromium picolinate, has been shown to improve insulin sensitivity and reduce elevated blood sugar and glycated hemoglobin levels and, as a result, reduce the risk of micro and macro vascular complications. Selenium reduces LDL oxidation and cytotoxicity

Optimized levels of taurine, camitine, arginine, leutine, lycopene, beta-carotene, niacin (vitamin B3), pantethine (a stable disulfide derivative of vitamin B5 (pantothenic acid)), vitamin C, particularly as dehydroascorbate, and vitamin E have also been found to be beneficial in improving cardiac function and in reducing the risk of cardiovascular disease. Gingko Biloba has been shown to reduce cholesterol. Taurine is one of the end products of methionine metabolism. The largest concentration of free amino acid in the heart comprises taurine. Taurine acts as an antioxidant in cardiac tissue and demonstrates an inotropic effect (strengthens the contraction of the heart muscle). It is also responsible, along with glycine, for conjugating cholesterol for excretion. Carnitine appears to increase HDL, lower total serum cholesterol, improve cardiac efficiency and exercise tolerance in cardiac patients and reduce post-infarction mortality. L-carnitine supplementation in MI patients reduces ventricular arrhythmias, reduces the extent of necrotic tissue, improves heart rate systolic pressure, decreases angina attacks, improves lipid pattern and significantly reduces mortality rate. Nitric oxide, necessary for vasodilation and regulation of vascular tone, is synthesized by the biometabolism of arginine resulting in increased blood flow and reduced blood pressure. Pantethine has beneficial effects on blood lipids, and reduces total cholesterol and triglycerides while increasing HDL cholesterol synthesis.

Curcumin is the active component of the plant/spice referred to as turmeric (Curcuma longa). The root and rhizome of turmeric have been used medicinally. The plant extract is standardized to 90-95% curcumin. It is a strong antioxidant, reduces insulin requirements, is a potent inhibitor of lipid peroxidation and participates in several anti-inflammatory mechanisms including the lowering of histamine levels and the potential of increasing natural cortisone production by the adrenals and modulating specific interleukins, cytokines, leukotrienes and eicosanoid synthesis in general. Curcumin has been shown to modulate many inflammatory markers such as TNF-a and NF-Kappa-b. It also provides hepatoprotective benefits against a number of toxic compounds. Recent studies indicate that curcumin also demonstrates anti-platelet effects which may protect against platelet aggregation and platelet adhesions, has shown anti-glycation benefits and has been shown to decrease platelet-activating factor (PAF) which disrupts normal platelet function. Supplementation with tumeric reduces oxidative stress and attenuates the development of fatty streaks in rabbits fed a high cholesterol diet.

Alpha Lipoic Acid (ALA), a disulfide molecule (a compound containing two thiol groups), is a unique antioxidant that is both lipid and water soluble and promotes synthesis of the endogenous antioxidant, glutathione. Studies indicate that ALA enhances glucose uptake and inhibits glycosylation. ALA has demonstrated the ability to prevent AGE induced increases in NF-kappa-b activation, thus protecting against endothelial dysfunction.

It was further found that AGEs induce lipid peroxidation in a neuronal cell line in a dose-dependant manner, and that blocking the specific AGE-receptor RAGE, as well as using different antioxidants (alpha-lipoic acid, N-acetylcysteine, 17 beta-estradiol or aminoguanidine) can reduce the AGE-mediated formation of lipid peroxidation products. Extracellularly administered alpha-lipoic acid reduces AGE-albumin-induced endothelial expression of VCAM-1 and monocyte binding to endothelium in vitro as well as demonstrating significant antioxidant potential.

L-Carnosine (b-alanyl-L-Histidine) is a naturally occurring di-peptide of the amino acids alanine and histidine. It is found in brain, muscle and other innervated tissues. High concentrations of carnosine are present in long-lived cells. Carnosine, a powerful antioxidant, is active against by-products and metabolites caused by reactive oxygen species as well as an anti-glycosylation effect. MDA (malondialdehyde), a marker of DNA damage from oxidative stress is blocked by carnosine. Carnosine both prevents sugar aldehydes from reacting with the amino acid on protein molecules as well as reversing the process.

Epigallocatechin-3-gallate (EGCG), recovered from green tea extract is a potent anti-inflammatory and antioxidant compound.

Piperine, a component of the spice black pepper, increases the bioavailability of curcurmin and epigallocatechin-3-gallate. Piperine also exhibits significant antioxidant activity of its own, as well as significant chemopreventative and immunomodulary effects.

Antioxidants, including Vitamins C, E, niacin and beta-carotene are known to protect LDL cholesterol from oxidative damage and thus reduce myocardial infarction and coronary artery disease. Generally 400 IU of vitamin E should be delivered on a daily basis. However, the quantity of alpha-tocopherol, which is one form of vitamin E, should be controlled because excess amounts can reduce the serum levels of other forms of vitamin E (Gamma- and delta tocopherol) as well as certain tocotrienols which also have unique health properties. There are seven different naturally occurring forms of vitamin E and tocotrienols. Niacin can reduce LDL and triglycerides and increase HDL cholesterol by reducing hepatic apolipoprotein A-1 clearance. However, pharmacological dosages of niacin (3,000 mg/day of nicotinic acid) has also been reported to increase homocysteine levels (Garg, R et al, Am. Heart J., 138, p 1082-7 (1999). Vitamin C has been shown to increase red blood cell glutathione, enhance vascular integrity reducing vascular permeability, increase HDL cholesterol and reduce total cholesterol.

### SUMMARY

A composition for treating, preventing or reducing the symptoms, causes and risk factors involved in cardiovascular diseases comprises a combination of ingredients. The primary ingredients are folate, phytosterols and flavones and may also include other ingredients which cooperate with these compounds or provide added benefits in addressing CVD. Particular preferred additional ingredients include the B vitamins, various natural antioxidants, particularly vitamin C and curcumin. Piperin when added appears to increase the efficiency of the various compositions.

### BRIEF DESCRIPTION OF DRAWINGS

FIG. 1, shown spread over two pages as FIG. 1a and FIG. 1b, is a diagram showing the interaction and participation of various natural compounds in normal metabolism.

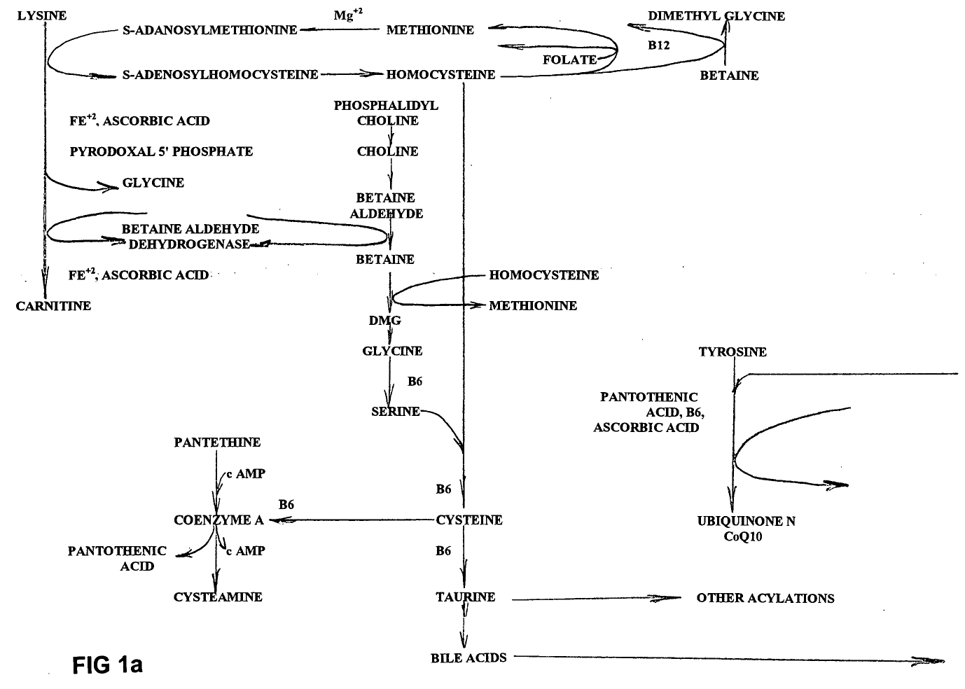

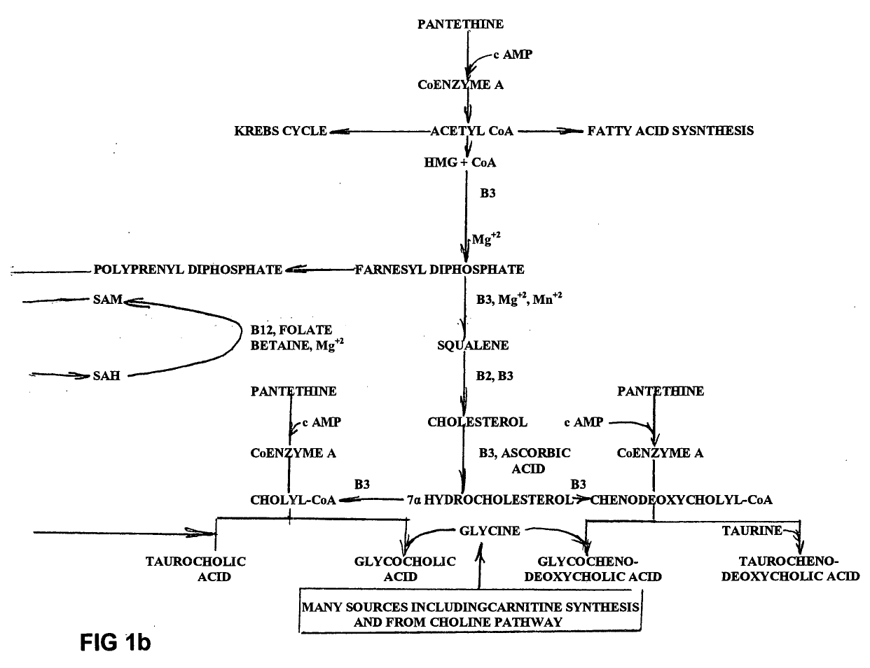

### DETAILED DESCRIPTION

Several major biochemical phenomena, pathways or risk factors, namely inflammation, oxidative stress, glycation/dysinsulinemia and elevated weight and body fat resulting in part there from, platelet function, endothelial function, LDL- and HDL-cholesterol levels (lowering LDL and elevating HDL), and elevated blood pressure and key markers, such as triglycerides, lipoprotein(a), fibrinogen, C-reactive protein (CRP) and homocysteine levels, are important contributors to the development or progression of cardiovascular disease. Lp(a) lipoprotein, a low density glycoprotein with apoliprotein B-100 linked to apoprotein(a), is a key factor in the development of arterial blockage by impairing fibrinolysis and, as a result increasing plaque buildup. Additionally, these risk factors are also important in reducing the risk of stoke.

While elevated LDL-cholesterol has been indicated as an independent risk factor, oxidized LDL levels can be of greater concern because the oxidized form causes endothelial damage and leads to atherosclerosis. While LDL receptors in the endothelium have a reduced uptake for oxidized LDL, oxidized LDL has a much greater tendency to stick to vessel walls than does LDL which is not oxidized, and microphages take up the oxidized LDL at a significantly greater rate forming engorged “foam cells” which embed themselves in the vascular endothelium to begin the formation of atherosclerotic plaque. The oxidized LDL also appears to be cytotoxic to endothelial cells. It appears that inflammation may play as big or a bigger role in cardiovascular disease as cholesterol. Statin drugs reduce inflammation moderately but they have no effect on homocysteine levels. A natural medicine that can demonstrate the ability to produce a 20-40% reduction in cholesterol levels as well as reduce causes of inflammation and reduce homocysteine levels would be a welcome addition to physician's treatment options for patients either with high cardiovascular risk factors or those already diagnosed with cardiovascular disease. Individual naturally occurring compounds generally cannot produce reductions of this magnitude. However, as discussed below, a combination of natural compounds can be effective in accomplishing this reduction while at the same time addressing other risk factors. Approximately 20% of the cholesterol in the body comes from food awhile the remaining 80% is produced by the liver (endogenous generation of cholesterol). While it is beneficial to reduce the amount of cholesterol absorbed through the gut from ingested food, it is more beneficial to interfere with the production of cholesterol by the body. This can be accomplished by decreasing apolipoprotein B, and diacylglycerol acetyl transferase and inhibiting HMG CoA reductase or increasing its breakdown.

Currently available phytosterols only generate cholesterol reductions of 10-15%, which is not enough reduction for individuals with high total cholesterol levels. Phytosterols reduce cholesterol by competing with cholesterol for absorption or blocking the absorption of cholesterol. However, phytosterols are hydrophobic and not readily soluble in water. Applicant has discovered that by surrounding the phytosterols in a system of naturally occurring hydrophilic compounds, thus increasing their solubility, the phytosterols are capable of reducing cholesterol levels more efficiently. Polymethoxylated flavones also reduce cholesterol but their mechanism is one that probably involves reducing normal cholesterol synthesis in the body rather than blocking its absorption.

Several naturally occurring compounds or group of compounds have been identified by applicant to decrease, reverse or prevent these physiological changes leading to or caused by CVD from occurring. While use of each separately is beneficial in treating cardiovascular disease applicant has discovered that there is a synergistic benefit in combining three or more of these compounds into a cocktail. Each compound address one or more of the different mechanisms or pathways which contribute to cardiovascular disease or reduce the markers associated with CD. Further, the combination creates an environment where it is difficult for atherosclerotic plaque to either develop or deposit and in which the levels of LDL-cholesterol are reduced.

Applicant has discovered that dietary supplementation with folate may be beneficial in treating and preventing several medical conditions. In particular, compositions set forth herein, which include folates, in combination with vitamin B6 and B12, particularly in the form of cyanocobalamin, hydroxyl cobalamin, S-adenosylocobalamin or methylcobalamine, have been found to be beneficial in preventing, reducing the severity of, or reversing various cardiovascular diseases, atherosclerosis and cardiac problems and risk factors which contribute to atherosclerosis and cardiovascular and cardiac problems including, but not limited to elevated blood pressure, fibrinogen and cholesterol, particularly low density lipoproteins, and sclerotic plaque buildup in the blood vessels even though the individual does not appear to have a folate or B12 deficiency or elevated homocysteine levels. These compositions may also be beneficial in preventing B12 deficiencies or elevated homocysteine levels. Also preferably included in the composition effective for treating the multiple factors relating to cardiovascular disease are phytosterols, preferably in a carrier to increase water solubility and polymethoxylated flavones. The water solubility of the phytosterols can be increased by microencapsulation, particularly in the form of liposomes, or blending with one or more of the dextrins suitable for food applications or in drug delivery formulation, particularly alpha, beta, gamma, hydroxypropyl, beta and methyl beta forms. It has also been found that a combination of the P5P form of vitamin B6, with vitamin C and/or curcumin is effective in reducing C-reaction protein levels and, in turn, reducing the risk of atherosclerosis by stopping LDL oxidation.

While a single cause for CVD has not been identified, the various causes or contributing factors are addressed by the invention.

Inflammation—Chronic inflammation damages host tissue. The invention described herein uses the therapeutic benefits of naturally occurring compounds to slow or halt the chronic inflammatory-like process that occurs in the early pathological cascade of CVD. Markers of inflammatory response include serum alpha (1) anti-chymotripsin, NF-kappaBeta, high sensitivity C-reactive protein, platelet activation factor, transforming growth factor beta, TNF-alpha and inflammatory cytokine production in general. Naturally occurring compounds (phytochemicals) that have a beneficial impact on inflammation can be beneficial in prevention of CVD and in slowing its progression, especially because many of these processes are measurable long before clinical symptoms appear.

Oxidative Stress—Like inflammation, oxidative stress plays a role in the development and progression of most chronic degenerative diseases of which CVD is no exception. Sources of oxidative stress are multiple and include advanced glycation end products and microglial activation. Membrane permeable antioxidants prevent the up-regulation of induced nitric oxide synthase (iNOS) and can be viewed both as antioxidants as well as anti-inflammatory drugs. Treatment with antioxidants is also beneficial in preventing and/or slowing CVD. Of particular interest are combinations of antioxidants that have complementary or synergistic activity or quench multiple types of reactive oxygen species.

Glycation/Dysinsulinemia—An increasing percentage of adults and children are overweight; obesity often causes dysinsulinemia that can lead to increased glycation of proteins. There is an increasing tendency toward Non-Insulin Dependent Diabetes Mellitus (NIDDM) even in people within normal body mass indicies (BMIs). This trend, coupled with the potential effects of glycation on CVD is of concern. Glycoxidative (glycation+oxidation) stress creates a cascade of events leading to CVD. The accumulation of advanced glycation end products (AGEs) explain pathological and biochemical events such as protein cross linking, free radical damage, neuronal apoptosis and glial activation that are features of AD. Several markers of glycoxidative stress have been identified. Examples of these markers are pentosidine, N(epsilon)-(carboxymethyl)lysine(CML), fructosamine, malondialdehyde(MDA), 4-hydroxy-2-noneal (HNE) which can be quantitatively measured in patients. Several naturally occurring ingredients (AGE Inhibitors), discussed below, can slow, halt or reverse glycoxidative effects on AD.

Platelet Function—Platelets are a source of beta-amyloid precursor protein. Increased platelet activation and increased circulating beta-amyloid has been identified with platelet aggregation and supports their adhesion. There is considerable in vitro evidence that non-steroidal anti-inflammatory drugs (NSAIDs) can reduce the inflammatory response of microglial cells. Ingredients that are both anti-inflammatory and normalize platelet function are beneficial as therapeutic options in CVD. Ingredients meeting both of these requirements, discussed below in regard to the therapeutic cocktail, have been found to be more effective when combined with other active compounds than single agents for CVD.

Metabolic Syndrome—A combination of four risk factors, high blood pressure, high insulin levels, excess body weight and abnormal cholesterol levels, has been referred to by the medical profession as metabolic syndrome. When this combination of factors exists there is a greater likelihood that an individual will develop diabetes, heart disease or stroke and each of these disorders is by itself a risk factor for other diseases. In combination, these disorders dramatically increase an individual's chance of developing potentially life-threatening illnesses. If one component of the metabolic syndrome exists, one or more of the other components are likely to be present; the more components present, the greater the risks to health. A study has shown that men with three of the metabolic syndrome factors are nearly twice as likely to have a heart attack or stroke and more than three times more likely to develop heart disease than those with none. The compositions set forth herein, and combinations of the various compounds which address each of these factors can significantly reduce the presence of metabolic syndrome and the incidence of diseases which have been related to this combination of factors.

The Cardiovascular Cocktail.

One composition for use in preventing, treating or reducing the severity of CVD comprises a combination of materials selected from B-vitamins (B-1, B-2, B-5, B-6, B-12 and folate), phytosterols, particularly phytosterols treated to increase their solubility by rendering them more hydrophilic, and flavonoids, preferably, polymethoxylated flavones from citrus extract. Other additives particularly beneficially when included in the composition are magnesium and/or calcium, β-carotene, vitamin C, curcumin, and piperine. Table 1 lists several additives which have benefits for inclusion in a cardiovascular formula for addressing the risk factors and markers of CVD. It should be noted as discussed below, several of these compounds address more then one of these factors. Further, there generally are no maximum daily dosage levels for these compounds or they are not toxic unless consumed in very high quantities and they are generally recognized to be safe for daily consumption.

TABLE 1
Medical Food Cocktail Ingredients and CVD Processes Targeted

| | EFFECT ON BIOCHEMICAL PROCESS AND RISK FACTORS | | | | | | | |
|---|---|---|---|---|---|---|---|---|
| | Oxidative | | | Platelet | Normal | | Endothelial | Blood |
| Ingredient | Stress | Inflamation | Glycation | Function | Metabolism | Cholesterol | Funct. | Press. |
| Curcumin | * | * | * | * | | * | * | |
| Piperine | * | * | * | * | | | | |
| EGCG | * | * | | * | | | | |
| ALA | * | * | * | * | | | | |
| N-Acetyl-cysteine | * | * | * | | | | | |
| Niacin (B3) | | | | | | * | | |
| B1 | | | * | | | | | |
| B6 | * | * | * | * | * | | | |
| B12 | * | * | | | * | | | |
| Folate | * | | | | * | | | |
| B2 | | | | | * | | | |
| B5 | | | | | * | | | |
| Vit. C | * | * | | | | * | | |
| Vit E | * | * | | * | | * | | |
| Vit. A | | * | | | | | | |
| SAMe | | | | | * | | | |
| Omega 3 Fatty Acid | | * | | | | | | |
| βCarotene | | * | | | | | | |
| Ginko Biloba | | | | | | * | | |
| CoQ10 | | | | | | * | | * |
| Phytosterols | | | | | | * | | |
| Flavonoids | * | | | * | | * | | |
| Mg | | | | | | * | | * |
| Ca | | | | | | * | | * |
| Chromium | | | * | | | | | |
| Selenium | * | | | | * | | | |
| Tocotrienol | * | * | | | * | | | |
| Policosanol | | | | * | | * | | |
| L-Carnitine | | | | | | * | | |
| Carnosine | * | | | | | | | |
| Taurine | * | | | | | * | | |
| Glycine | | | | | | * | | |
| Argenine | | | | | * | | | |
| Pantethine | | | | | | * | | |
| Inositol hexaniacinate | | | | | | * | | |

Applicant has found that certain lesser known metabolites or alternative forms of some of the B vitamins, such as B-1, B-6 and B-12, play important roles beyond their identified uses for reduction of homocysteine. For example, the hydroxycobalamine form of B-12 has been found to scavenge NO radicals. The benfotiamine form of vitamin B-1 has demonstrated significant benefit against excessive glycation and advanced glycation end products (AGEs) which have been associated with glia inflammation and folate compositions have now been found to address inflammation, for example caused by NO, as well as endothelial function. Nitrogen oxide (NO) synthase creates NO which is inflammatory to tissue. The beneficial properties of folates can also be enhanced by the concurrent use of certain B vitamins, particularly pyridoxal-5-phosphate (P5P) pyridoxamine and hydrocobalamin, and antioxidants, such as vitamin E, SAMe and CoQ10. Addition of NO synthase inhibitors, such as amino-guanidine, carnosine, asymmetric argentine, and certain plant derived phytochemicals can enhance the inflammation reducing properties of folates. A preferred daily dosage comprises 100 mcg. to 15 mg, preferably 400 mcg to 10 mg, of folate along with one or more of the hydroxycobalamin form of B12 (100 mcg-1 mg), B6 (pyridoxal-5-phosphate (P5P) or pyridoxamine) (1 mg to 100 mg), and 25 mg-1,000 mg of riboflavin (B2) along with phytosterols and flavones. When present, preferred dosages include about 500 mg of NAC, 100 mg of EGCG from green tea extract, 300 mg of alpha lipoic acid, 1,000 mg of turmeric (95% curcumin), 25 mg of vitamin B2, 25 mg of vitamin B1 (benfotiamine), 300 mg of vitamin C and 400 IU of tocopheryl succinate (vitamin E) and at least about 2.5 mg of piperin.

### EXAMPLE 1

A first composition comprises folate and one or more of vitamin B6, vitamin B12 and polymethoxylated flavones as active ingredients and may also include other active ingredients such as magnesium salts, for example magnesium stearate, MgO, magnesium citrate, etc., as well as inert carriers which comprise the delivery system, such as rice flour, gelatin and silicon dioxide. A particular preferred combination of active ingredients for a single pill or capsule is set forth in Table 2. A single pill, capsule, or other form of delivery includes 150 mg of polymethoxylated flavone, along with the other listed ingredients. A preferred single dosage is two pills or capsules (300 mg polymethoxylated flavones) and a preferred daily dosage (in a 24 hour period) is 2-4 pills or capsules (300-600 mg of polymethoxylated flavones per day).

TABLE 2

| Active Ingredients | Concentration Per Unit | |
|---|---|---|
| Citrus Extract (30% | 500 | mg |
| polymethoxylated flavones) | (150 mg polymethoxylated flavones) | |
| Pyridoxal-5-phosphate | 25 | mg |
| Folic acid | 2 | mg |
| Methylcobalamin | 500 | mcg |

### EXAMPLE 2

A second composition comprises phytosterol and one or more of β-carotene, folate, vitamin B12 and magnesium as active ingredients. The formulation may also include inert carriers which comprise the delivery system such as tocopherol, caprylic acid, safflower oil, water, methylparaben, lecithin and sorbitol oleate. It has been found that by encapsulating the phytosterol, for example in liposomes or in dextrins, the solubility of ingested phytosterols, normal having low water solubility, can be significantly increased. As a result, much less of the encapsulated phytosterols need be delivered to provide an effective amount. That same quantity delivered with out encapsulations would generally be ineffective as it is not readily absorbed by the body. A particularly preferred combination of active ingredients for a single pill or capsule is set forth in Table 3. A preferred single dose is two capsules (300 mg phytosterol) with said single dose preferably delivered one or twice a day (300-600 mg phytosterol per day)

TABLE 3

| Active Ingredients | Concentration | |
|---|---|---|
| Phytosterol | 150 | mg |
| Magnesium (as an oxide) | 30 | mg |
| Folic acid | 2.5 | mg |
| β-carotene | 2.5 | mg |
| Methylcobalamin | 500 | mcg |

A particularly preferred phytosterol containing composition per pill or capsule is listed in Table 4. The primary constituents are an encapsulated phytosterol, the phytosterol being encapsulated by a medium chain triglyceride, preferably caprylic/capric triglycerides, with a small quantity of hydroxylated lecithin. A single dose would comprise 2 pills or capsules (containing 300 mg phytosterol) and a preferred daily dosage is 300 mg-600 mg phytosterol with the second 300 mg being delivered 8-16 hours after the first 300 mg.

TABLE 4
CAPSULE COMPOSITION*

| | Quantity, mg | Composition % |
|---|---|---|
| Active Ingredients | | 25.0 |
| Phytosterol | 150 | 24.6 |
| β-Carotene | 2.5 | 0.4 |
| MgO | 40 | 6.55 |
| Liquid Excipients | | |
| Caprylic/ | 152.5 | 25.0 |
| Capric Triglycerides | | |
| Safflower Oil | 90 | 14.75 |
| Purified water | 48.25 | 7.9 |
| Sorbital Oleate | 100.25 | 16.47 |
| Tocopherol | 1.5 | 0.25 |
| Solid Excipients | | |
| Methylparaben | 0.5 | 0.08 |
| Polyparaben | 0.25 | 0.04 |
| NaCl | 9.0 | 1.47 |
| Hydroxylated Lecithin | 15.0 | 2.5 |
| Total Weight, mg | 610 | |

*Normal dose is two capsules (300 mg Phytosterol) delivered twice a day

A third preferred composition would be a combination of the compositions of Example 2 and 3, containing as the primary constituents folate, vitamins B6 and B12, encapsulated phytosterol and polymethoxylated flavones. Shown in Table 5 is the composition for a single capsule or pill; a daily dosage would be 2-4 capsules or pills of the Table 5 composition.

TABLE 5

| Active Ingredients | Concentration | |
|---|---|---|
| Phytosterol | 150 | mg |
| Magnesium (as an oxide) | 30 | mg |
| Folic acid | 2.5 | mg |
| β-carotene | 2.5 | mg |
| Methylcobalamin | 500 | mcg |
| Citrus Extract (30% | 500 | mg |
| polymethoxylated flavones) | (150 mg polymethoxylated flavones) | |
| Pyridoxal-5-phosphate | 25 | mg |

The compositions of Examples 1-5 may also include one or more of the other constituents listed in Table 1, or derivatives or metabolites thereof. While specific formulations or combinations of compounds have been set forth as beneficial or preferred, the invention is not limited to those combinations, or compositions listed herein as other compounds, whether natural, or synthesized may be discovered to be active in treating or preventing cardiovascular diseases. The invention is limited only by the claims set forth herein which preferably include folate in combination with other active ingredients for prevention, or treatment or reduction of the symptoms of CVD.

It is preferred that these compositions be delivered orally and the components be prepared for ingestion in a manner that makes the composition available in therapeutically effective amounts. As such, they may be prepared as water soluble compositions, delivered in liquid form, lyophilized, encapsulated, or in a manner suitable for time release, delayed release or enteric delivery, or any manner typically used for orally delivered pharmaceuticals, nutraceuticals or vitamins, or combined with foods or other normally ingested products. However, the invention is not limited to oral delivery as the compositions set forth herein may also be delivered by nasal spray, inhalation techniques, transdermally, transmucossal, by suppository, injected or by intravenous methods.

A preferred method of encapsulating the ingredients, particularly the phytosterols which are insoluble or only sparingly soluble in water, is to encapsulate the constituent or constituents in lipid liposomes or dextrins or combinations thereof. Suitable dextrins include, but are not limited to, alpha, beta, gamma, hydroeypropylbeta, methyl beta, etc. U.S. Pat. Nos. 6,726,924 and 6,610,322, incorporated herein by reference, are examples of techniques which can be used to encapsulate the components, combinations of one or more of the components or the entire composition set forth herein, and particularly to encapsulate phytosterols. While encapsulation of the phytosterols is particularly beneficial because of the low solubility of the sterols, even more soluble constituents, such as the isoflavones, curcumin, etc., can be beneficial in increasing their solubility and, in turn, absorption by the body and efficiency as a disease preventive or treatment. Still further, this beneficial effect is not limited to delivery of the compounds or compositions listed herein for treatment of CVD but also extends to treatment of other diseases which benefit from delivery of the specific compounds, for example, curcumin delivered to treat Alzheimers and other neurological disorders and migraine treatment both addressed in applicants prior filed patent applications referenced above.

I claim:

1. A composition for preventing, treating or reducing the symptoms of, or risk factors relating to, cardiovascular disease or atherosclerosis comprising a combination of compounds effective for reducing or preventing two or more of oxidative stress, glycation or glycosylation and inflammation and normalizing platelet function, cholesterol levels, blood pressure, and homocysteine levels.

2. The composition of claim 1 comprising at least:
a) folate and one or more of vitamin B2, vitamin B6, vitamin B12, or metabolites or derivatives thereof, and
b) phytosterols.

3. The composition of claim 1 comprising a daily dose of at least three compounds selected from:
a) at least about 100 mcg folate and one or more of at least about 25 mg of vitamin B2, at least about 1 mg of vitamin B6 in the form of pyridoxal-5-phosphate or pyridoxamine, at least about 100 mcg of the hydroxycobalamin form of vitamin B12, or metabolites or derivatives thereof, and
b) at least about 300 mg of phytosterols.

4. The composition of claim 3 wherein the phytosterol is microencapsulated in a phospholipid or carried by a dextrin.

5. The composition of claim 4 wherein the dextrin is chosen from the group consisting of the alpha, beta, gamma, hydroxypropyl beta, and methyl beta forms of dextrin.

6. The composition of claim 2 further including a flavone.

7. The composition of claim 6 wherein the flavone is a polymethoxylated flavone.

8. The composition of claim 2 or claim 6 further including piperin.

9. The composition of claim 1 comprising folate, polymethoxylated flavones and one or more of vitamin B6 and vitamin B12.

10. The composition of claim 8 containing about 2.5 mg folic acid, about 25 mg of the pyridoxal-5-phosphate form of vitamin B6, about 500 mcg of the methylcobalamin form of vitamin B12 and about 150 mg of polymethoxylated flavone from citrus extract.

11. The composition of claim 1 comprising phytosterol, magnesium, α-carotene, folate, and vitamin B12.

12. The composition of claim 11 comprising about 150 mg of an encapsulated phytosterol, about 30 mg of magnesium as magnesium oxide, about 2.5 mg of β-carotene, about 2.5 mg of folic acid and about 500 mcg of the methylcobalamin form of vitamin B12.

13. The composition of claim 1 comprising
a) folate and one or more of vitamin B6 and vitamin B12
b) a phytosterol, and
c) a polymethoxylated flavone.

14. The composition of claim 2, claim 9, or claim 13 further including one or more of Curcumin, Piperine, EGCG, ALA, N-Acetylcysteine, niacin (B3), vitamins B2, B5, B6, B12, folate, vitamin C, E, A, SAMe, Omega 3 Fatty Acid, βCarotene, Ginko Biloba, COQ10, Phytosterols, Flavonoids, Mg, Ca, Chromium, Selenium, Tocotrienol, Policosanol, L-Carnitine, Carnosine, Taurine, Glycine, Argenine, Pantethine, and Inositol hexa-niacinate.

15. The composition of claim 9 further including one or more of Curcumin, Piperine, EGCG, ALA, N-Acetylcysteine, niacin, vitamins A, B1, B2, B5 C, and E, SAMe, Omega 3 Fatty Acid, βCarotene, Ginko Biloba, COQ10, Phytosterols, Flavonoids, Mg, Ca, Chromium, Selenium, Tocotrienol, Policosanol, L-Carnitine, Carnosine, Taurine, Glycine, Argenine, Pantethine, and Inositol hexa-niacinate.

16. The composition of claim 13 further including one or more of Curcumin, Piperine, EGCG, ALA, N-Acetylcysteine, niacin, vitamins A, B1, B2, B5 C, and E, SAMe, Omega 3 Fatty Acid, βCarotene, Ginko Biloba, CoQ10, Flavonoids, Mg, Ca, Chromium, Selenium, Tocotrienol, Policosanol, L-Carnitine, Carnosine, Taurine, Glycine, Argenine, Pantethine, and Inositol hexa-niacinate.

17. The composition of claim 1 comprising folate, any two of the P5P form of vitamin B, vitamin C and curcumin.

18. A process for treating, preventing or reducing metabolic syndrome comprising delivering to an individual a composition comprising at least
a) folate and one or more of vitamin B2, Vitamin B6, Vitamin B12, or metabolites or derivatives thereof, and one or more of
b) phytosterol and a flavone.

19. A composition for preventing, treating or reducing the symptoms of, or risk factors relating to, cardiovascular disease or atherosclerosis comprising phytosterol, β-carotene and magnesium as active ingredients in an encapsulating carrier.

20. The composition of claim 19 wherein the active ingredients comprise about 25%_{w} of the active ingredients and about 75%_{w} of the encapsulating carrier.

21. The composition of claim 20 wherein the encapsulating carrier comprises
a) caprylic or capric triglycerides, safflower oil, water, sorbitol oleate and tocopherol as liquid excipients, and
b) methylparaben, paraben, NaCl, and lecithin as solid excipients.

22. The composition of claim 21 comprising about 2.5% by weight of lecithin.

23. The composition of claim 19 wherein the encapsulating carrier includes a dextrin.

24. The composition of claim 22 wherein the dextrin is chosen from the group consisting of the alpha, beta, gamma, hydroxypropyl beta, and methyl beta forms of dextrin.

25. The composition of claim 19 wherein a single dosage comprises about 300 mg of phytosterol and a daily dosage comprises about 300 mg to about 600 mg of phytosterol in a 24 hour period.

26. The composition of claim 25 wherein a single dosage comprises two capsules, each containing about 150 mg of phytosterol.
